(19) United States
(12) Patent Application Publication
(10) Pub. No.: US 2018/0030432 A1
(43) Pub. Date: Feb. 1, 2018

(54) DEVICE FOR HANDLING OF MAGNETIC PARTICLES AND METHOD FOR HANDLING MAGNETIC PARTICLES

(71) Applicants: Shimadzu Corporation, Kyoto (JP); KAZUSA DNA RESEARCH INSTITUTE, chiba (JP)

(72) Inventors: Masaki KANAI, Kyoto (JP); Hiroyuki JIKUYA, Kyoto (JP); Tetsuo OHASHI, Kyoto (JP); Shin NAKAMURA, Kyoto (JP); Osamu OHARA, Chiba (JP)

(73) Assignees: Shimadzu Corporation, Kyoto (JP); KAZUSA DNA RESEARCH INSTITUTE, Chiba (JP)

(21) Appl. No.: 15/547,216

(22) PCT Filed: Jan. 30, 2015
(86) PCT No.: PCT/JP2015/052686

§ 371 Date: Jul. 28, 2017

(51) Int. Cl. C12N 15/10; B03C 1/28; B01J 19/08

(52) U.S. Cl. CPC C12N 15/1013; B01J 19/087; B03C 1/28; B01J 2219/085; B01J 2219/089; B03C 2201/18

(57) ABSTRACT

A device for handling of magnetic particles, in which liquids and a gel-like medium are loaded. The device is provided with: a first liquid containing part in which a first liquid is contained; a second liquid contained, part in which a second liquid is contained, a third liquid containing part in which a third liquid is contained, and a first gel-like medium containing part in which the first gel-like medium is contained. The first liquid containing part, the second liquid containing part and the third liquid containing part are connected to the first gel-like medium containing part. The first liquid, the second liquid and the third liquid are separated from each other by the first gel-like medium.

### TECHNICAL FIELD

The present invention relates to a device for handling of magnetic particles and a method for handling magnetic particles for performing chemical handling such as separation, extraction, purification, or reaction of a target substance by using magnetic particles.

### BACKGROUND ART

In medical examination, food safety and hygiene management, monitoring for environmental preservation, or the like, it is required to extract a target substance from a sample containing various kinds of contaminants and provide the target substance for detection and reaction. For example, in medical examination, in some cases, it is necessary to detect, classify, and quantify nucleic acids, proteins, sugars, lipids, bacteria, viruses, radioactive substances, or the like contained in blood, serum, cells, urine, feces or the like separated and acquired from animals and plants. In the examination, in some cases, it is necessary to separate and purify the target substance in order to eliminate adverse influences of background or the like caused by contaminants.

In order to separate and purify a target substance in a sample, a method of using magnetic particles provided with a chemical affinity with the target substance and a molecular recognition function on surfaces of magnetic substances having a particle size of about 0.5 μm to about several tens of μm has been developed and put to practical use. In this method, processes of immobilizing the target substance on the surfaces of the magnetic particles, after that, separating and recovering the magnetic particles from a liquid phase by magnetic field handling, and if necessary, dispersing the recovered magnetic particles in a liquid phase such as a cleaning liquid, and separating and recovering the magnetic particles from the liquid phase are repeatedly performed. After that, by dispersing the magnetic particles in an eluting liquid, the target substance immobilized to the magnetic particles is separated in the eluting liquid, and the target substance in the eluting liquid is recovered. By using the magnetic particles, since the recovering of the target substance with a magnet is possible, the technique has features that it is advantageous for automation of chemical extraction and purification.

Magnetic particles capable of selectively immobilizing a target substance are commercially available as a portion of a separation/purification kit. In the kit, a plurality of reagents are contained in different containers, and at the time of using the reagents, a user dispenses and pipettes the reagents with a pipette or the like. A device for automating such a pipetting operation or magnetic field handling is also commercially available.

On the other hand, a method of separating and purifying a target substance by moving magnetic particles along the longitudinal direction of a tubular container in a tubular device by using the tubular device where a liquid layer (liquid phase) such as a dissolving/immobilizing liquid, a cleaning liquid, an eluting liquid, and the like and a gel-like medium layer (a gel-like medium phase) are alternately laminated in a tubular container such as a capillary instead of the pipetting operation has been disclosed (refer to Patent Literature 1). In addition, a method of separating and purifying a target substance by moving magnetic particles along the longitudinal direction of a groove in a chip device by using the chip device in which a liquid phase and a gel-like medium phase are alternately arranged in the groove formed in a surface of a substrate has also been disclosed (refer to Patent Literature 2).

### CITATION LIST

### Patent Literatures

Patent Literature 1: International Publication No. 2012/086243

Patent Literature 2: JP-A-2013-130548

### DISCLOSURE OF THE INVENTION

### Problem to be Solved by the Invention

In the tubular device as disclosed in Patent Literature 1 and the chip device as disclosed in Patent Literature 2, the liquid phase and the gel-like medium phase are alternately arranged in each of the devices, and the liquids are separated by a gel-like medium. For this reason, when various kinds of the liquids are to exist in the device, it is necessary to allow a large amount of the gel-like medium for separating the liquid to exist, and thus, the work of loading the liquids and the gel-like medium becomes complicated. In particular, when the gel-like medium is to be loaded, contamination easily occurs due to the gel-like medium adhering to an inner wall of the device, and in order to prevent this contamination, in the device of the related art, a tube or a groove cannot be excessively thinned.

Furthermore, when various kinds of liquids are to exist in the device, it is necessary to lengthen the tubes and the grooves, so that the size of the device becomes large.

As described above, in the device of the related art, in a case where various kinds of liquids are to exist, there is room for improvement in the manufacturing or size of the device.

In view of the above, the invention is to provide a device for handling of magnetic particles which can easily load a liquid and a gel-like medium and can reduce the size of the device even in a case where various kinds of liquids exist in the device.

### Means for solving problem

As a result of studies, the present inventors have found that, by using a device having a gel-like medium containing part connected to three or more liquid containing parts, even in a case where various kinds of liquids exist in the device, it is easy to load a liquid and a gel-like medium and it is possible to reduce the size of the device, and the present inventors have completed the invention.

The invention relates to a device for handling of magnetic particles loaded with a liquid and a gel-like medium. The device includes a first liquid containing part containing a first liquid, a second liquid containing part containing a second liquid, a third liquid containing part containing a third liquid, and a first gel-like medium containing part containing a first gel-like medium. Each of the first liquid containing part, the second liquid containing part, and the third liquid containing part is connected to the first gel-like medium containing part, and the first liquid, the second liquid, and the third liquid are separated by the first gel-like medium. The first liquid, the second liquid, and the third liquid may not be different kinds of liquids or may contain the same kind of the liquid.

The device may further include a fourth liquid containing part containing a fourth liquid, and the fourth liquid containing part may be connected to the first gel-like medium containing part.

In one embodiment, the device includes only the first gel-like medium containing part as a gel-like medium containing part containing a gel-like medium.

The device may further include a fourth liquid containing part containing a fourth liquid and a second gel-like medium containing part containing a second gel-like medium. In one embodiment, each of the third liquid containing part and the liquid containing part is connected to the second gel-like medium containing part, and the third liquid and the fourth liquid are separated by the second gel-like medium. The first gel-like medium and the second gel-like medium may not be different kinds of gel-like media or may be the same kind of gel-like medium.

It is preferable that the first liquid containing part, the second liquid containing part, the third liquid containing part, and the first gel-like medium containing part have outer wall surfaces formed on the same plane.

It is preferable that the magnetic particles to be moved in the device are loaded into the device.

The invention relates to a kit for manufacturing the above-described device for handling of magnetic particles.

The invention relates to a method for handling magnetic particles for moving magnetic particles in the above-described device for handling of magnetic particles. The method according to the invention includes steps of moving the magnetic particles in a first liquid containing part to a first gel-like medium containing part by magnetic field handling; moving the magnetic particles in the first gel-like medium containing part to a second liquid containing part by magnetic field handling; moving the magnetic particles in the second liquid containing part to the first gel-like medium containing part by magnetic field handling; and moving the magnetic gel particles in the first gel-like medium containing part to a third liquid containing part by magnetic field handling. In addition, which of the liquid containing parts is to be the first liquid containing part, the second liquid containing part, or the third liquid containing part is determined by the kind of the liquid contained in the liquid containing part. In addition, in a case where the same kind of the liquid is contained in a plurality of liquid containing parts, the order of moving the magnetic particles to these liquid containing parts is not limited. For this reason, even in a device using a container having the same shape, it is possible to arbitrarily set the order of moving the magnetic particles.

### Effect of the Invention

According to the device for handling of magnetic particles of the invention, even in a case where various kinds of liquids exist in the device, it is easy to load the liquids and a gel-like medium, and it is possible to reduce the size of the device.

### BRIEF DESCRIPTION OF THE DRAWINGS

FIG. 1 is a schematic perspective view illustrating an embodiment of a device for handling of magnetic particles according to the invention.

FIG. 2 is a cross-sectional view of the device for handling of magnetic particles illustrated in FIG. 1.

FIG. 3 is a schematic cross-sectional view illustrating an embodiment of a device for handling of magnetic particles including a plurality of gel-like medium containing parts.

FIG. 4 is a schematic cross-sectional view illustrating an arrangement of liquid containing parts.

FIG. 5 is a schematic perspective view illustrating an embodiment of a device for handling of magnetic particles according to the invention.

### MODE FOR CARRYING OUT THE INVENTION

### [Device for Handling of Magnetic Particles]

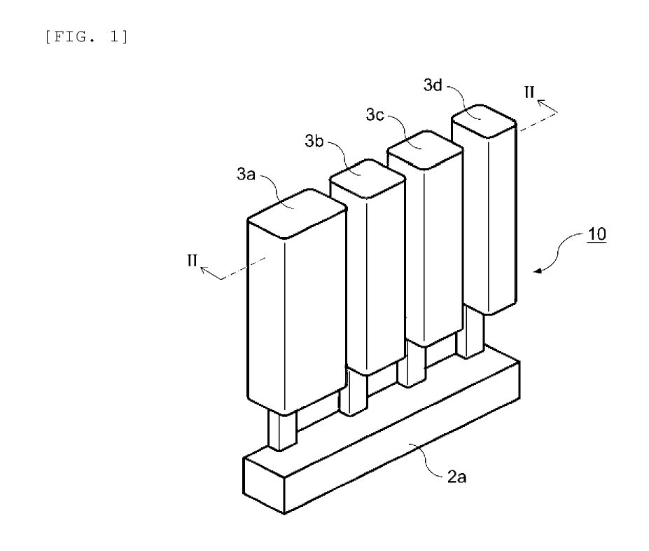

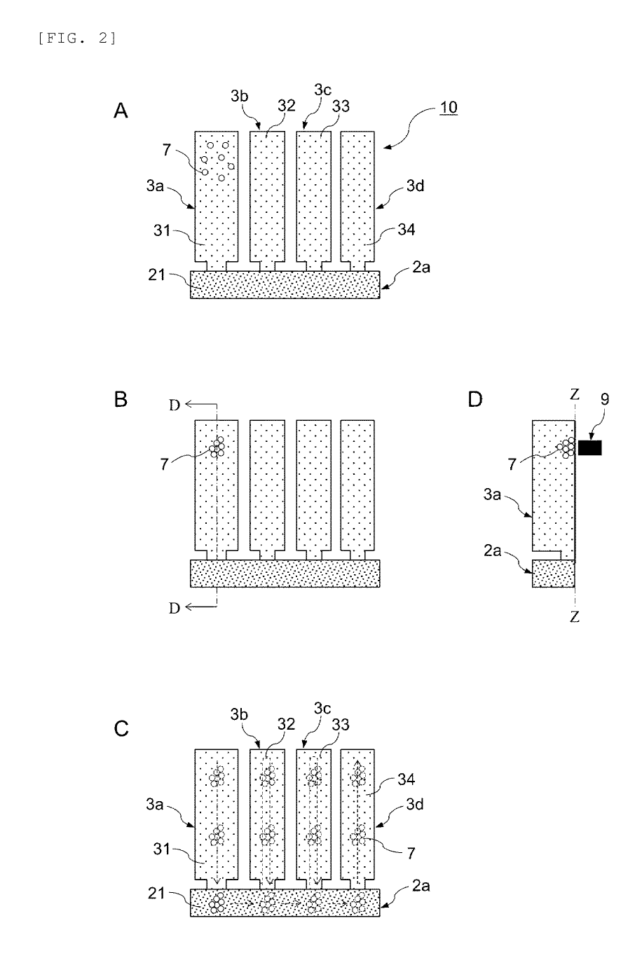

FIG. 1 is a schematic perspective view illustrating an embodiment of a device for handling of magnetic particles according to the invention (hereinafter, also simply referred to as a device), and FIGS. 2A to 2C are cross-sectional views of the device taken along line II-II of FIG. 1. FIG. 2D is a cross-sectional view taken along line D-D of FIG. 2B.

As illustrated in FIGS. 1 and 2A, the device 10 is configured to include a liquid containing part 3a containing a liquid 31, a liquid containing part 3b containing the liquid 32, a liquid containing part 3c containing a liquid 33, a liquid containing part 3d containing a liquid 34, and a gel-like medium containing part 2a containing a gel-like medium 21.

The liquid containing part 3a, the liquid containing part 3b, the liquid containing part 3c, and the liquid containing part 3d are connected to the gel-like medium containing part 2a, respectively. The gel-like medium is not miscible with the liquid in the adjacent liquid containing part and is insoluble or hardly soluble in the liquid. Therefore, the liquid 31, the liquid 32, the liquid 33 and the liquid 34 are separated by the gel-like medium 21.

In FIG. 2A, the liquid 31 of the liquid containing part 3a contains a large number of magnetic particles 7. The magnetic particles 7 are particles capable of specifically immobilizing target substances such as nucleic acids and antigens on the surface or inside thereof. By dispersing the magnetic particles 7 in the liquid 31, the target substance contained in the liquid 31 is selectively immobilized to the particles 7.

As illustrated in FIG. 2D, when a magnet 9 serving as a magnetic force source is brought close to the outer wall surface of the liquid containing part 3a, the magnetic particles 7 to which the target substance is immobilized are magnetically attracted to the liquid containing part 3a (refer to FIGS. 2B and 2D).

If the magnet 9 is sequentially moved along the outer wall surfaces of the liquid containing part 3a, the gel-like medium containing part 2a, the liquid containing part 3b, the gel-like medium containing part 2a, the liquid containing part 3c, the gel-like medium containing part 2a, and the liquid containing part 3d, the magnetic particles 7 are also moved following the change of the magnetic field, so that the magnetic particles are sequentially moved in order of the liquid 31, the gel-like medium 21, the liquid 32, the gel-like medium 21, the liquid 33, the gel-like medium 21, and the liquid 34 (refer to FIG. 2C). Most of the liquids as liquid droplets physically adhering to the surroundings of the magnetic particles 7 are desorbed from the particle surfaces when the magnetic particles enter the inside of the gel-like medium. Although the gel-like medium is perforated by the penetration and movement of the magnetic particles into the gel-like medium 21, the holes of the gel-like medium are immediately blocked due to a self-repairing action by a restoring force of the gel. For this reason, there occurs almost no flow of liquids into the gel-like medium through through-holes formed by the magnetic particles.

As illustrated in FIG. 1 and FIG. 2D, the liquid containing part 3a and the gel-like medium containing part 2a have outer wall surfaces formed on the same plane (Z-Z cross-section in FIG. 2D). As illustrated in FIG. 1, the liquid containing parts 3b, 3c, and 3d also have outer wall surfaces formed on the same plane. If each of the liquid containing part and the gel-like medium containing part has the outer wall surface formed on the same plane, the magnet 9 can be easily moved along the outer wall surface, so that the magnetic particles can be smoothly moved. As described above, although it is preferable that each of the liquid containing part and the gel-like medium containing part has the outer wall surface formed on the same plane, as long as the magnetic particles can be moved, the shape of the outer wall surface is particularly limited.

In the device according to the invention having the above-described configuration, unlike the device of the related art in which the liquid and the gel-like medium are alternately arranged, the liquids are separated by the common gel-like medium (the gel-like medium 21 in FIGS. 2A to 2C). Accordingly, even in a case where various kinds of liquids (liquids 31 to 34 in FIGS. 2A to 2C) exist in the device, it is easy to load the liquids and the gel-like medium, and it is possible to reduce a problem of contamination that easily occurs particularly at the time of loading the gel-like medium.

Furthermore, since each of the liquid containing parts for storing the respective liquids is connected to the gel-like medium containing part, even when various kinds of liquids exist in the device, there is no need to manufacture an elongated device like a tubular device of the related art where the liquid and the gel-like medium are alternately arranged. Therefore, it is possible to load the liquid and the gel-like medium into the device without using a nozzle or the like.

In addition, in the device of the related art, it has been difficult to individually change the sizes (shapes, volumes, or the like) of the portion where the liquids are loaded and the portions where the gel-like mediums are loaded, but in the device according to the invention, since the containing part and the gel-like medium containing part are independent, the sizes of the liquid containing part and the gel-like medium containing part can be arbitrarily set.

In FIG. 2C, in the direction in which the magnetic particles are moved in the liquid containing part, the magnetic particles are allowed to be moved from the upper portion to the lower portion inside the liquid containing part 3a, the magnetic particles are allowed to be moved from the lower portion to the upper portion inside the liquid containing parts 3b and 3c, and the magnetic particles are allowed to be moved from the lower portion to the upper portion inside the liquid containing part 3d. However, as long as the magnetic particles can be dispersed in each liquid, the direction in which the magnetic particles are moved in the liquid containing part is not particularly limited.

In FIG. 2C, although the magnetic particles 7 are moved in order of the liquid 31, the liquid 32, the liquid 33, and the liquid 34, the order of moving the magnetic particles 7 is not particularly limited, and the order is determined depending on the kinds of the liquids contained in the liquid containing parts. For example, by exchanging the kinds of the liquids contained in the liquid containing parts 3a and 3b, the magnetic particles 7 may be moved in order of the liquid 32, the liquid 31, the liquid 33, and the liquid 34. In addition, in a case where the liquid 32 and the liquid 33 are the same kinds of liquids (for example, a cleaning liquid or the like), the magnetic particles 7 may be moved in order of the liquid 31, the liquid 32, the liquid 33, and the liquid 34 or may be moved in order of the liquid 31, the liquid 33, the liquid 32, and the liquid 34. Therefore, in the device according to the invention, even in a device using a container having the same shape, the order of moving the magnetic particles can be arbitrarily set.

As described above, in the device according to the invention, unlike tubular devices or chip devices of the related art where the magnetic particles are allowed to be moved only in one direction, since the order of moving the magnetic particles can be freely set according to the arrangement of the liquid containing parts or the like, various processes can be implemented.

Furthermore, by using the device according to the invention, it is possible to easily recover plural kinds of solutions obtained by the handling using magnetic particles. As described later, in the handling using the magnetic particles, it is possible to elute the target substances immobilized to the magnetic particles into the liquid, and for example, the target substances is immobilized on the surfaces of the magnetic particles in the first liquid containing part, and the target substances are eluted in a low salt concentration solution in the second liquid containing part. After that, the magnetic particles are moved to the third liquid containing part, and the target substances are eluted in a higher salt concentration solution. In this case, by recovering the solutions in the second liquid containing part and the third liquid containing part, it is possible to easily produce a low salt concentration elution fraction and a high salt concentration elution fraction by a series of operation. Although it is difficult to perform such handling in a tubular device of the related art where a liquid and a gel-like medium are alternately arranged, in the device according to the invention, it is possible to easily realize the handling by forming a solution discharge port in each liquid containing part.

Although FIGS. 2A to 2C illustrate an example where the four liquid containing parts 3a to 3d are connected to the gel-like medium containing part 2a, the number of the liquid containing parts connected to the gel-like medium containing part 2a may be three or more and may be three or five or more.

FIGS. 2A to 2C illustrate an example where the four liquid containing parts 3a to 3d are connected only to the gel-like medium containing part 2a, that is, an example where the device includes only one gel-like medium containing part. However, in the device according to the invention, as long as the device includes a gel-like medium containing part (first gel-like medium containing part) connected to three or more liquid containing parts, the other gel-like medium containing part (second gel-containing part) may be further included. In this case, it is preferable that the second gel-like medium containing part is connected to the liquid containing part connected to the first gel-like medium containing part.

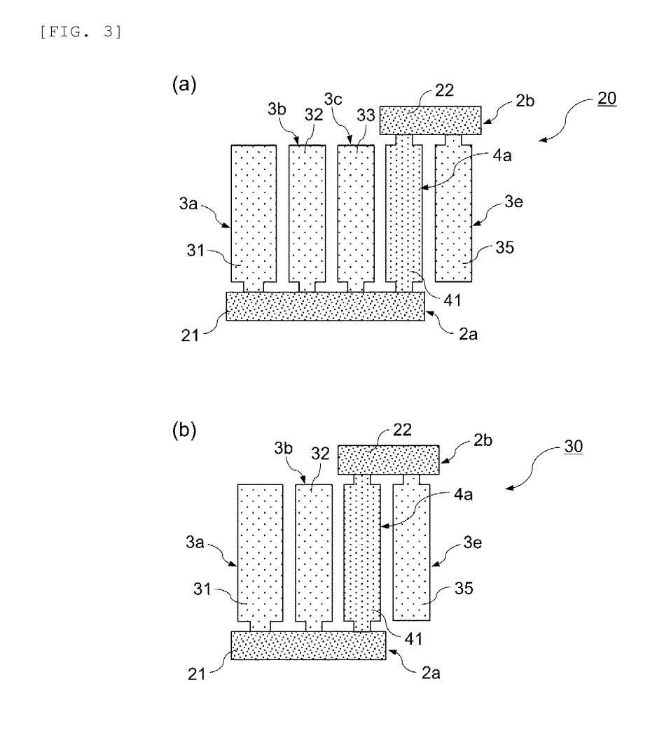

FIGS. 3(a) and 3(b) are schematic cross-sectional views illustrating an embodiment of a device for handling of magnetic particles including a plurality of gel-like medium containing parts. The device 20 illustrated in FIG. 3(a) is configured to include a liquid containing part 3a containing a liquid 31, a liquid containing part 3b containing a liquid 32, a liquid containing part 3c containing a liquid 33, a liquid containing part 4a containing a liquid 41, a liquid containing part 3e containing a liquid 35, a gel-like medium containing part 2a containing a gel-like medium 21, and a gel-like medium containing part 2b containing a gel-like medium 22. Each of the liquid containing part 3a, the liquid containing part 3b, the liquid containing part 3c, and the liquid containing part 4a is connected to the gel-like medium containing part 2a. Each of the liquid containing part 4a and the liquid containing part 3e is connected to the gel-like medium containing part 2b. Therefore, in FIG. 3(a), the liquid 31, the liquid 32, the liquid 33, and the liquid 41 are separated by the gel-like medium 21, and the liquid 41 and the liquid 35 are separated by the gel-like medium 22.

In a case where the device according to the invention includes a plurality of gel-like medium containing parts, like the device 30 illustrated in FIG. 3(b), the number of the liquid containing parts connected to the gel-like medium containing part 2a may be three. In addition, the number of liquid containing parts connected to the gel-like medium containing part 2a may be five or more.

The number of the liquid containing parts connected to the gel-like medium containing part 2b is not limited to two, and three or more liquid containing parts may be connected to the gel-like medium containing part 2b. In addition, the liquid containing part (liquid containing part 4a in FIGS. 3(a) and 3(b)) connected to the plurality of gel-like medium containing parts is not limited to one, and two or more liquid containing parts may be connected to a plurality of gel-like medium containing parts.

Although FIGS. 3(a) and 3(b) illustrate an example where there is one gel-like medium containing part other than the gel-like medium containing part 2a, that is, an example where the device includes two gel-like medium containing parts, the device may include three or more gel-like medium containing parts. In this case, the number of the liquid containing parts connected to each of the gel-like medium containing parts is not particularly limited, and the numbers may be the same or different from each other.

In some cases, according to the kind of the liquid, the liquid may permeate into the gel-like medium. For this reason, in a case where the device according to the invention includes a plurality of gel-like medium containing parts, it is possible to use the device such that a liquid containing part containing a liquid which easily permeates into a specific gel-like medium (for example, the first gel-like medium) is connected to a second gel-like medium containing part containing a gel-like medium (for example, the second gel-like medium) into which it is difficult for the liquid to permeate, and the other liquid containing part is connected to the first gel-like medium containing part.

The device according to the invention may further include a gel-like medium containing part connected to only one liquid containing part. For example, the device illustrated in FIGS. 2A to 2C may include a gel-like medium containing part connected only to the liquid containing part 3a. The same configuration is applied to the liquid containing parts 3b to 3d.

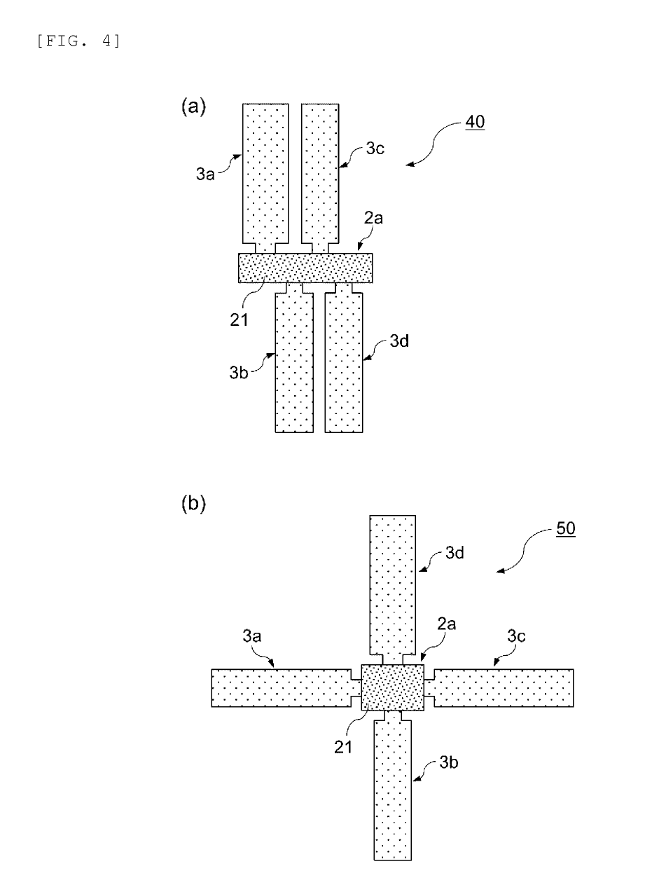

Although, in the above description, the embodiment where the liquid containing parts are connected to the same surface of the gel-like medium containing part (the upper surface of the gel-like medium containing part 2a in FIGS. 2A to 2C) has been described, the arrangement of the liquid containing parts is not particularly limited. For example, like the device 40 illustrated in FIG. 4(a), the liquid containing parts 3a and 3c may be connected to the upper surface of the gel-like medium containing part 2a, and the liquid containing parts 3b and 3d may be connected to the lower surface of the gel-like medium containing part 2a. In addition, like the device 50 illustrated in FIG. 4(b), the liquid containing parts 3a to 3d may be radially connected around the gel-like medium containing part 2a.

In the device according to the invention, particularly in a case where various kinds of liquids exist in the device, the size of the whole device can be easily adjusted by setting the liquid containing parts to a desired arrangement.

The shapes of the liquid containing parts are not particularly limited, and examples thereof include a tubular shape and a groove shape as described later. The shapes of the respective liquid containing parts may be the same or different from each other.

The thickness of the liquid containing part is not particularly limited. If the thickness of the liquid containing part is constant on the side facing the magnet, the distance between the magnet and the inner wall surface of the liquid containing part can be maintained constant, so that the magnetic particles can be moved smoothly. For this reason, it is preferable that the thickness of the liquid containing part is constant on the side facing the magnet.

The length of the liquid containing part is not particularly limited, and for example, the length maybe about 5 mm to 50 mm. As described above, unlike a device in the related art in which the liquid and the gel-like medium are alternately arranged, even in a case where various kinds of liquids exist in the device, since it is not necessary to lengthen the device, it is possible to reduce the size of the entire device.

The cross-sectional areas of the liquid containing parts are not necessarily the same, and when viewed in the longitudinal direction, a portion having a large cross-sectional area or a portion having a small cross-sectional area may exist. For example, FIG. 2A and the like illustrate an example where the cross-sectional area of the connecting portion with respect to the gel-like medium containing part is smaller than the cross-sectional areas of the other portions. In addition, although, in FIG. 2A and the like, the liquid is loaded into the connecting portion (portion having a relatively small cross-sectional area) between the liquid containing part and the gel-like medium containing part, the gel-like medium may be loaded into this portion.

In the plane perpendicular to the longitudinal direction of the liquid containing part, the cross-sectional area of the inner wall surface of the connecting portion between the liquid containing part and the gel-like medium containing part is preferably 0.2 mm² to 80 mm², more preferably 1.5 mm² to 25 mm².

The cross-sectional area, length, and the like of the inner wall of the liquid containing part may be selected appropriately according to the amount of the substance to be treated, the amount of the magnetic particles, and the like.

The shape and length of the gel-like medium containing part are not particularly limited as long as three or more liquid containing parts can be connected. In a case where a plurality of gel-like medium containing parts exist, the shapes thereof may be the same or may be different from each other. In addition, although the thickness of the gel-like medium containing part is not particularly limited, like the liquid containing part, it is preferable that the thickness of the gel-like medium containing part is constant on the side facing the magnet.

The container constituting the above-described device can be manufactured by a known method. For example, as a container constituting the device 10 illustrated in FIG. 1, a container including the tubular liquid containing parts 3a to 3d and the gel-like medium containing part 2a can be manufactured by a blow molding method or the like.

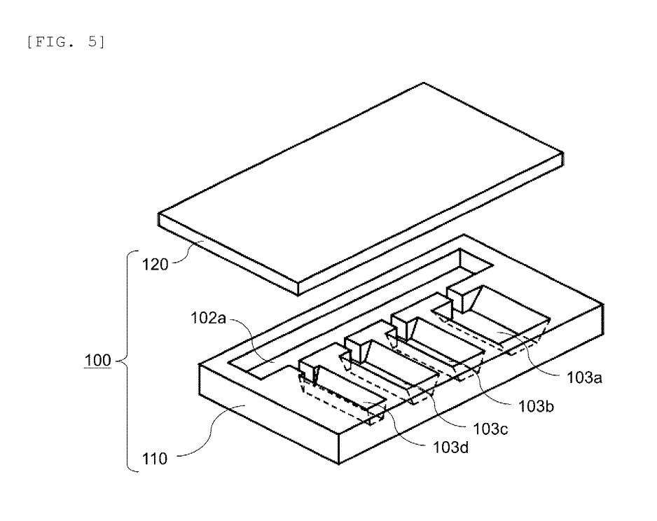

In addition, as a portion of the container constituting the device 100 illustrated in FIG. 5, the substrate 110 where grooves corresponding to the liquid containing parts 103a to 103d and the gel-like medium containing part 102a are formed can be manufactured by an injection molding method, a molding method, or the like. FIG. 5 illustrates the device 100 before the liquid and the gel-like medium are loaded, and a container constituting the device 100 can be manufactured by providing a cover plate 120 on the substrate 110 so as to cover the groove.

In FIG. 5, a hole communicating with the liquid contained in the liquid containing part may be drilled in the cover member 120. The hole can function as a sample supply port and a sample colletion port.

Although, in FIG. 5, the distal ends in the longitudinal direction of the grooves corresponding to the liquid containing parts 103a to 103d (the distal ends on the side opposite to the gel-like medium containing part 102a) are formed so as to be located inside the end face of the substrate 110, the grooves may be formed so that the distal ends thereof reach the end face of the substrate 110. In this case, opening portions are provided on the end face of the substrate, and the opening portions can be used as sample supply ports or sample discharge ports.

In the device according to the invention, the materials of the liquid containing parts and the gel-like medium containing parts are not particularly limited as long as the magnetic particles can be allowed to move in the device and the liquids and the gel-like medium can be retained. The materials of the liquid containing part and the gel-like medium containing part may be the same or different from each other, but it is preferable that the materials are the same. In order to move the magnetic particles in the device by handling of the magnetic field from the outside of the device, a magnetically permeable material such as plastic is preferred, and there may be exemplified resin materials of polyolefins such as polypropylene and polyethylene, fluorocarbon resins such as tetrafluoroethylene, cyclic polyolefins such as polyvinyl chloride, polystyrene, and polycarbonate, and the like. As a material of the liquid containing part and the gel-like medium containing part, a ceramic, a glass, silicon, a non-magnetic metal, or the like may be used besides the above-described materials. In order to enhance water repellency of the inner wall surface, coating with a fluorine resin, silicone, or the like may be performed.

In a case where optical measurements of absorbance, fluorescence, chemiluminescence, bioluminescence, refractive index change, or the like are performed during the handling of the particles or after the handling of the particles, or in a case where light irradiation is performed, it is preferable that the materials of the liquid containing part and the gel-like medium containing part have optical transparency. In addition, when the materials of the liquid containing part and the gel-like medium containing part have the optical transparency, it is preferable from the viewpoint that the state of particle handling in the device can be visually confirmed. On the other hand, in a case where it is necessary to shield the liquids, the magnetic particles or the like from light, it is preferable that the materials of the liquid containing part and the gel-like medium containing part do not have the optical transparency but the light-shielding property. It maybe divided into a light transmitting portion and a light shielding portion depending on the purpose of use and the like.

In the device according to the invention, as long as three or more liquid containing parts are connected to the gel-like medium containing part and the respective liquids are separated by the gel-like medium, other configurations are not particularly limited.

The method of immobilizing the target substance to the magnetic particles is not particularly limited, and various known immobilization mechanisms such as physical adsorption and chemical adsorption can be applied. The target substance is immobilized on the surface or inside of the particle by various intermolecular forces such as van der Waals force, hydrogen bonding, hydrophobic interaction, ionic interaction, and π-π stacking.

The particle size of the magnetic particles is preferably 1 mm or less, more preferably 0.1 to 500 μm. Although the shape of the particles is preferably spherical with a uniform particle size, irregular shapes with some degree of particle size distribution may be used as long as particle handling is possible. The constituent of the particle may be a single substance, or the particle may be made of a plurality of constituents.

Although the magnetic particles may be made of only a magnetic material, the magnetic particles provided with coating for specifically immobilizing the target substance on the surface of the magnetic material are preferably used. As a magnetic material, there may be exemplified iron, cobalt, nickel, and compounds, oxides, alloys, and the like thereof. More specifically, there maybe exemplified magnetite (Fe₃O₄), hematite (Fe₂O₃ or αFe₂O₃), maghemite (γFe₂O₃), titanomagnetite (xFe₂TiO₄(1-x)Fe₃O₄), ilmenohematite (xFeTiO₃(1-x)Fe₂O₃), pyrrhotite (Fe₁₋ₓS(x=0 to 0.13) Fe₇S₈ (x to 0.13)), greigite (Fe₃S₄), goethite (αFeOOH), chromium oxide (CrO₂), permalloy, alconi magnet, stainless steel, samarium magnet, neodymium magnet, and barium magnet.

As a target substance selectively immobilized on the magnetic particles, there may be exemplified a substance derived from a living body such as a nucleic acid, a protein, a sugar, a lipid, an antibody, a receptor, an antigen, and a ligand or a cell itself. In a case where the target substance is a substance derived from a living body, the target substance may be immobilized inside the particle or on the particle surface by molecular recognition or the like. For example, in a case where the target substance is a nucleic acid, magnetic particles provided with silica coating on the surface thereof are preferably used as magnetic particles. In a case where the target substance is an antibody (for example, a labeled antibody), a receptor, an antigen, a ligand or the like, the target substance can be selectively immobilized to the particle surface by an amino group, a carboxyl group, an epoxy group, avidin, biotin, digoxigenin, protein A, protein G, or the like on the particle surface. As magnetic particles capable of selectively immobilizing a specific target substance, commercially available products such as Dynabeads (registered trademark) sold by Life Technologies and MagExtractor (registered trademark) sold by Toyobo, or the like may also be used.

In FIGS. 2A to 2C, handling such as immobilization of the magnetic particles to the target substance by dispersing the magnetic particles 7 in the liquids 31 to 34 to allow the magnetic particles to be in contact with the liquids in the liquid containing parts, a cleaning operation for removing contaminants adhering to the surfaces of the magnetic particles, a reaction of the target substance immobilized to the magnetic particles, elution of the target substance immobilized to the magnetic particles into the liquid, and the like are performed.

For example, in a case where separation and extraction of nucleic acids are performed by using silica particles provided with silica coating, the magnetic particles 7 are dispersed in the liquid sample 31 containing a nucleic acid extracted liquid and nucleic acids, the nucleic acids are immobilized on the surfaces of the magnetic particles 7, and after that, the magnetic particles 7 are moved into the cleaning liquids 32 and 33. After dispersing the magnetic particles 7 in the cleaning liquids 32 and 33 and removing contaminating proteins adhering to the surfaces thereof, the magnetic particles 7 are moved into the nucleic acid eluted liquid 34. By dispersing the magnetic particles 7 in the nucleic acid eluted liquid 34, it is possible to recover the nucleic acid immobilized on the particle surfaces in the nucleic acid eluted liquid 34. In addition, although, in FIGS. 2A to 2C illustrate an example of a device including two liquid containing parts 3b and 3c as liquid containing parts where a cleaning liquid is loaded, the number of liquid containing parts where a cleaning liquid is loaded may be one or may three or more. In addition, the cleaning liquid can be omitted for the purposes of separation or as long as undesirable inhibition in the application does not occur.

In a case where the substance selectively immobilized on the magnetic particles is an antigen, by immobilizing the antigen in the liquid 31 as the first medium on the surfaces of the magnetic particles 7 coated with molecules capable of selectively immobilizing antigens such as Protein G and Protein A and dispersing the magnetic particles in the liquids 32 and 33 and by performing cleaning for removing contaminants adhering to the particle surfaces and dispersing the magnetic particles in the liquid 34 as the second medium, an antigen-antibody reaction between the antigens immobilized on the particle surfaces and the antibodies in the liquid 34, release and elution of the target substance into the liquid 34, and the like can be performed.

Since the above-described method for handling the particles need not generate a liquid flow with a pipette or the like, the method can be performed in a closed system. If the liquids, the gel-like media, and the magnetic particles are sealingly loaded into the container contamination from the outside can be prevented. For this reason, it is particularly useful in a case where an easily decomposable target substance such as RNA is immobilized to the magnetic particles to be operated or in a case where a liquid that easily reacts with oxygen or the like in the air is used. In a case where the container is a closed system, the container can be sealed by a method of thermally fusing an opening portion of the container or by using an appropriate sealing means. In a case where it is necessary to extract the particles after the handling and the liquid after the elution of the target substance to the outside of the container, it is preferable to seal the opening portion removably by using a resin stopper or the like. In addition, by arranging a gel-like medium or the like so as to be in contact with the liquid, the liquid may be sealingly loaded.

The liquid loaded into the container provides a site for chemical handling such as extraction, purification, reaction, separation, detection, or analysis of the target substance immobilized on the surfaces of the magnetic particles. The kind of the liquid is not particularly limited, but it is preferable that the liquid does not dissolve the gel-like medium. For this reason, as the liquid, an aqueous solution or a water-based liquid such as a mixed solution of water and an organic solvent is preferably used. Besides functioning merely as a medium for the above-described chemical handling, the liquid may directly participate in the chemical handling or may contain a compound involved in the handling as a component. As a substance contained in the liquid, there may be exemplified substances that react with reactive substances immobilized to the magnetic particles, substances that further react with substances immobilized on the surfaces of the magnetic particles by the reaction, reaction reagents, fluorescent substances, various kinds of buffers, surfactants, salts, various other adjuvants, organic solvents such as alcohols, and the like. The water-based liquid may be provided in an arbitrary form of water, an aqueous solution, and water suspension.

In the case of immobilizing the target substance contained in the liquid sample on the surfaces of the magnetic particles, in some cases, besides the target substance to be immobilized on the surfaces of the magnetic particles, various contaminants maybe included in the liquid. The liquid sample may contain biological samples of animal and plant tissues, body fluids, or excrement, nucleic acid including entities such as cells, protozoans, fungi, bacteria, viruses, or the like. The body fluids include blood, cerebrospinal fluid, saliva, milk, or the like, and the excrement includes feces, urine, sweat, or the like. The cells include leukocytes or blood platelets in blood, detached cells of mucosal cells such as oral cells, leukocytes in saliva, and the like.

A liquid sample containing a target substance such as a nucleic acid, an antigen, or an antibody may be produced in a form of, for example, a cell suspension, a homogenate, a mixed solution with a cell lysate, or the like. In a case where a target substance contained in a sample such as blood derived from a living body is to be immobilized on the particle surfaces, the liquid sample is a mixture of sample such as blood derived from the living body and the cell lysate (nucleic acid extracted liquid) for extracting the target substance therefrom. The cell lysate contains components such as chaotropic substances and surfactants capable of dissolving the cells.

The gel-like medium loaded into the container may be gel-like or paste-like before the particle handling. It is preferable that the gel-like medium is insoluble or sparingly soluble in the adjacent liquid and is a chemically inactive substance. Here, the term “insoluble or sparingly soluble in a liquid” denotes that the solubility in a liquid at 25° C. is about 100 ppm or less. The term “chemically inactive substance” denotes a substance that does not have a chemical influence on liquids, magnetic particles, or substances immobilized to the magnetic particles in contacting with the liquid or in handling of the magnetic particles (that is, handling for moving the magnetic particles in the gel-like medium).

The material, composition, and the like of the gel-like medium are not particularly limited, and the gel-like medium may be a physical gel or a chemical gel. For example, as disclosed in WO2012/086243, a water-insoluble or sparingly water-soluble liquid substance is heated, a gelling agent is added to the heated liquid substance, the gelling agent is completely dissolved, and after that, the substance is cooled down to a sol-gel transition temperature, so that a physical gel is formed.

As a chemical gel, there may be used hydrocarbon gels such as polyethylene, polystyrene, polypropylene, polyvinyl chloride, and (meth)acrylic polymer; silicone gels such as polysiloxane, PDMS, and silicone hydrogel; fluorine-based gels such as PTFE, PFA, FEP, ETFE, and PCTFE; and a gel-like or paste-like mixture containing the above-described gel as a main component. As a specific example of the hydrocarbon-based gel, there may be exemplified Plastibase (registered trademark) or the like containing polyethylene as a main component.

A chemical gel is one in which a plurality of polymer chains are crosslinked through covalent bonds by a chemical reaction, and thus, a gel state can be retained as long as the crosslinked structure is maintained. For this reason, the gel state is retained even after the magnetic particles pass through the gel-like medium. When the particles pass through the chemical gel medium, the gel is temporarily perforated, but the perforation is repaired instantaneously by the restoring force of the gel. For this reason, the components derived from the gel adhere to the surfaces of the magnetic particles, so that contaminants are rarely taken out of the gel. Therefore, by using a chemical gel as a gel-like medium, it is possible to improve the accuracy of purification and detection of the target substance by handling of particles. In addition, in the case of using a chemical gel, it is not necessary to perform gelling in the container, so that it is easy to load the gel into the container. Since the stability of a chemical gel is high, it is difficult for sol gelation to occur even by a physical action such as vibration during transportation and storage of the gel after the gel is loaded or by heating during exposure to a high temperature environment. For this reason, even in a case where there is provided a device in the state that the liquid and the gel-like medium are loaded in advance into the container, it is possible to enhance stability during transportation and storage of the device.

Among the chemical gels, a silicone gel is preferably used. As a polymer constituting the silicone gel, there may be exemplified crosslinked organopolysiloxanes such as crosslinked organopolysiloxane, alkyl-modified partially-crosslinked organopolysiloxane, and silicone-branched alkyl-modified partially-crosslinked organopolysiloxane. As an organopolysiloxane, dimethicone, vinyl dimethicone, methyl trimethicone, methylvinylsiloxane, lauryl dimethicone, copolymers thereof or the like is used. The molecular structure of the polymer is not particularly limited, but the molecular structure may be a straight chained structure, a branched straight chained structure, a cyclic structure, or a reticular structure. The silicone gel is obtained by swelling a polymer (or oligomer) of the above-described crosslinked organopolysiloxane in an oil agent. An oil agent which is obtained by swelling the above-described polymer is not miscible with a water-based liquid is appropriately used. As an oil agent, there may be exemplified cyclopentasiloxane, cyclomethicone, dimethicone, dimethiconol, methyl trimethicone, phenyl trimethicone, cyclopentasiloxane, diphenylsiloxyphenyl trimethicone, mineral oil, isododecane, isododecyl neopentanoate, trioctanoin, squalane, and the like. For example, a gel-like or paste-like silicone gel can be obtained by mixing fine particles of a polymer of a crosslinked organopolysiloxane with an oil agent.

A silicone gel in which a crosslinked organopolysiloxane is swollen in an oil agent is a chemical gel having a crosslinked structure and having a viscosity. For this reason, the silicone gel can easily pass the magnetic particles, and even when the gel is temporarily perforated, the silicone gel is immediately repaired, and thus, in the handling using the magnetic particles, the silicone gel is suitable as a gel-like medium for separating the liquid layers.

The loading of the gel-like medium and the liquid into the container can be performed by an appropriate method. For example, in a case where both the liquid containing part and the gel-like medium containing part are tubular, after the gel-like medium is loaded from an opening portion formed at one end of the liquid containing part into the gel-like medium containing part, each liquid may be loaded into each liquid containing part, or after each liquid is loaded from an opening portion formed in the gel-like medium containing part into in each liquid containing part, the gel-like medium may be loaded into the gel-like medium containing part. In addition, in the case of a device including a substrate and a cover plate, the gel-like medium is loaded into the site corresponding to the gel-like medium containing part among the grooves formed on the surface of the substrate, and after that, the liquid may be loaded into the site corresponding to the liquid containing part.

The amounts of the gel-like medium and the liquid loaded into the container can be appropriately set according to the volumes of the liquid containing part and the gel-like medium containing part, the amount of the magnetic particles to be operated, the type of the handling, and the like. As described above, in a case where a plurality of gel-like medium containing parts are provided in the device, the volumes of the respective gel-like medium containing parts may be the same or different from each other. The volumes of the respective liquid containing parts may be the same or different from each other.

The device for handling of magnetic particles according to the invention can be manufactured by loading a gel-like medium and a liquid into a container including a tubular liquid containing part and a gel-like medium containing part having the above-described shapes. In addition, the device can be manufactured by loading a gel-like medium and a liquid into a container including a substrate and a cover plate having grooves having the above-described shape.

The liquid to be loaded into the container is, for example, a liquid such as a nucleic acid extracted liquid capable of dissolving cells. This liquid may be one to which alcohol or the like is added. The magnetic particles are loaded into the container at the time of using the device. In addition, the device may be produced in a state in which a liquid such as a nucleic acid extracted liquid and magnetic particles coexist in advance.

### [Kit for Manufacturing Device for Handling of Magnetic Particles]

Apart from the container, a gel-like medium and a liquid or the like may be independently provided. The loading of the gel-like medium and the liquid into the container may be performed immediately before the handling of the magnetic particles or may be performed with a sufficient time before the handling of the magnetic particles. In a case where the gel-like medium is insoluble or sparingly soluble in the liquid, even when a long period of time has elapsed after the loading, almost no reaction or absorption occurs between the gel-like medium and the liquid.

The magnetic particles may be provided as a component of a kit for manufacturing a device. The magnetic particles may be provided as a component of the kit in a state that the magnetic particles coexist in the liquid.

The amount of magnetic particles contained in the device or in the kit is appropriately determined depending on the type of the chemical handling to be targeted, the volumes of the liquid containing part and the gel-like medium containing part, and the like. For example, in a case where the cross-sectional area of the connecting portion between the liquid containing part and the gel-like medium containing part is about 2 mm² to 15 mm², the amount of magnetic particles is usually preferably in a range of about 10 to 200 μg.

### [Example of Handling of Particles]

As described above, in the handling using the magnetic particles, by repeating the dispersion of the magnetic particles in the liquid and the movement of the magnetic particles into the other liquid, separation, purification, reaction, detection, and the like of the target substance are performed. For example, in a case where nucleic acids are separated and extracted by using the magnetic particles provided with silica coating, the magnetic particles are dispersed in a sample containing nucleic acids, the nucleic acids are immobilized on the surfaces of the magnetic particles, and after that, the magnetic particles are moved into the cleaning liquid. The magnetic particles are dispersed in the cleaning liquid, the contaminating proteins and the like adhering to the surface are removed, and after that, the magnetic particles are moved into the nucleic acid eluted liquid. The magnetic particles are moved into the nucleic acid eluted liquid. By dispersing the magnetic particles in the nucleic acid extracted liquid, it is possible to recover the nucleic acids immobilized on the particle surfaces in the eluted liquid.

As a cell lysate (nucleic acid extracted liquid) used for extracting the nucleic acids, there may be exemplified a chaotropic substance, a chelating agent such as EDTA, and a buffer solution containing tris hydrochloride, or the like. In addition, the cell lysate may also contain a surfactant such as Triton X-100. As a chaotropic substance, there may be exemplified guanidine hydrochloride, guanidine isothiocyanate, potassium iodide, urea, and the like. In addition to the above-described materials, the cell lysate may contain proteolytic enzymes such as protease K, various buffers, salts, various other adjuvants, organic solvents such as alcohols, and the like.

The cleaning liquid may be obtained by separating a component (for example, protein, carbohydrate, or the like) other than the nucleic acids contained in the sample, a reagent used for treatment such as nucleic acid extraction, or the like in the cleaning liquid in a state that the nucleic acids are immobilized on the particle surfaces. As the cleaning liquid, there may be exemplified a high salt concentration aqueous solution of sodium chloride, potassium chloride, ammonium sulfate and the like, an aqueous alcohol solution of ethanol, isopropanol, and the like.

As a nucleic acid eluted liquid, there may be used a buffer solution containing water or a low concentration salt. More specifically, a tris buffer solution, a phosphate buffer solution, distilled water, or the like can be used, and a 5 to 20 mM tris buffer solution adjusted to pH 7 to 9 is generally used. By dispersing the magnetic particles immobilized with the nucleic acids in the eluted liquid, it is possible to separate and elute the nucleic acids in the nucleic acid eluted liquid. The recovered nucleic acids can be subjected to handling such as concentration and drying as necessary and, after that, can be provided to analysis, reaction, or the like.

In addition, in a case where ELISA (enzyme-linked immuno-sorbent assay) is performed, magnetic particles immobilized with primary antibodies are used, and in a first liquid containing test antigens (test substances), the primary antibodies immobilized to the magnetic particles are reacted with the test antigens. As a result, the antigens to be detected in the liquid are selectively immobilized to the surfaces of the magnetic particles. After the magnetic particles are cleaned in the second liquid, the antigen-antibody reaction between the enzyme-labeled secondary antibodies and the test antigens immobilized on the surfaces of the magnetic particles is performed in the third liquid. Therefore, the secondary antibodies are immobilized on the surfaces of the magnetic particles through the primary antibodies on the surfaces of the magnetic particles and the test antibodies. After the magnetic particles are cleaned in the fourth liquid, a coloring reaction between the enzyme bound to the secondary antibodies immobilized on the particle surfaces in a fifth liquid and the chromogenic substance is performed for a certain period of time. Quantitative evaluation can be performed by monitoring the color reaction by spectrophotometric absorbance measurement. In addition, in the case of qualitative evaluation, the coloring reaction may be visually confirmed.

After the coloring reaction is performed for a certain period of time in the fifth liquid, the magnetic particles may be moved from the fifth liquid to a sixth liquid. By moving the magnetic particles to the outside of the fifth liquid, the coloring reaction can be stopped. For this reason, since the quantitative evaluation can be performed without stopping the coloring reaction by newly adding a reaction stopping reagent such as sodium hydroxide, even in a case where the fifth liquid is hermetically sealed, quantitative measurement can be performed.

As described above, in the case of performing the ELISA, since the reaction and cleaning are repeated, by sequentially moving the magnetic particles, the magnetic particles are dispersed in each liquid. In the case of performing the ELISA, since many kinds of liquids are required as compared with the case of performing separation/extraction of nucleic acids, it is possible to appropriately use the device according to the invention.

### EXPLANATIONS OF LETTERS OR NUMERALS

10, 100 device for handling of magnetic particles
2a, 2b, 102a gel-like medium containing part
3a, 3b, 3c, 3d, 3e, 4a, 103a, 103b, 103c, 103d liquid containing part
21, 22 gel-like medium
31, 32, 33, 34, 35, 41 liquid
7 magnetic particles
9 magnets

1-10. (canceled)

11. A device for handling of magnetic particles loaded with a liquid and a gel-like medium, comprising:
a first liquid containing part containing a first liquid;
a second liquid containing part containing a second liquid;
a third liquid containing part containing a third liquid; and
a first gel-like medium containing part containing a first gel-like medium,
wherein each of the first liquid containing part, the second liquid containing part and the third liquid containing part is connected to the first gel-like medium containing part, and
wherein the first liquid, the second liquid, and the third liquid are separated by the first gel-like medium.

12. The device for handling of magnetic particles according to claim 11, further comprising a fourth liquid containing part containing a fourth liquid,
wherein the fourth liquid containing part is connected to the first gel-like medium containing part.

13. The device for handling of magnetic particles according to claim 11, comprising only the first gel-like medium containing part as a gel-like medium containing part containing a gel-like medium.

14. The device for handling of magnetic particles according to claim 11, further comprising a fourth liquid containing part containing a fourth liquid and a second gel-like medium containing part containing a second gel-like medium,
wherein each of the third liquid containing part and the fourth liquid containing part is connected to the second gel-like medium containing part, and
wherein the third liquid and the fourth liquid are separated by the second gel-like medium.

15. The device for handling of magnetic particles according to claim 11, wherein the first liquid containing part, the second liquid containing part, the third liquid containing part, and the first gel-like medium containing part have outer wall surfaces formed on the same plane.

16. The device for handling of magnetic particles according to claim 11, wherein the magnetic particles to be moved in the device are loaded into the device.

17. A kit for manufacturing the device for handling of magnetic particles according to claim 11, comprising:
a container which includes a first liquid containing part containing a first liquid, a second liquid containing part containing a second liquid, a third liquid containing part containing a third liquid, and a first gel-like medium containing part containing a first gel-like medium and in which each of the first liquid containing part, the second liquid containing part, and the third liquid containing part is connected to the first gel-like medium containing part; and
a gel-like medium which is to be contained in the first gel-like medium containing part.

18. The kit for manufacturing the device for handling of magnetic particles according to claim 17, further comprising liquids, each of which is to be contained in each of the first liquid containing part, the second liquid containing part, and the third liquid containing part.

19. A method for handling magnetic particles for moving the magnetic particles in a device loaded with liquids, a gel-like medium and magnetic particles,
wherein the device includes a first liquid containing part containing a first liquid, a second liquid containing part containing a second liquid, a third liquid containing part containing a third liquid, and a first gel-like medium containing part containing a gel-like medium,
wherein each of the first liquid containing part, the second liquid containing part, and the third liquid containing part is connected to the first gel-like medium containing part,
wherein the first liquid, the second liquid, and the third liquid are separated by the first gel-like medium, and
wherein the method for handling the magnetic particles comprising steps of:
moving the magnetic particles in the first liquid containing part to the first gel-like medium containing part by magnetic field handling;
moving the magnetic particles in the first gel-like medium containing part to the second liquid containing part by magnetic field handling;
moving the magnetic particles in the second liquid containing part to the first gel-like medium containing part by magnetic field handling; and
moving the magnetic particles in the first gel-like medium containing part to the third liquid containing part by magnetic field handling.

20. The method for handling magnetic particles according to claim 19,
wherein the first liquid containing part, the second liquid containing part, the third liquid containing part, and the first gel-like medium containing part have outer wall surfaces formed on the same plane, and
wherein the magnetic particles are moved along the outer wall surfaces.
